(19) United States
(12) Patent Application Publication
(10) Pub. No.: US 2026/0056182 A1
(43) Pub. Date: Feb. 26, 2026

(54) System

(71) Applicant: Softbank Group Corp., Tokyo (JP)

(72) Inventor: Seiwa Kobayashi, Tokyo (JP)

(21) Appl. No.: 19/308,824

(22) Filed: Aug. 25, 2025

(30) Foreign Application Priority Data
Aug. 26, 2024 (JP) 2024-144820

(51) Int. Cl. G01N 33/24; G06Q 50/04

(52) U.S. Cl. CPC G01N 33/245; G01N 33/246; G06Q 50/04

(57) ABSTRACT

A system comprises a processor that is configured to receive measurement data from a sensor device configured to measure soil condition, water content, and light intensity, store the measurement data in a database, analyze the stored data to generate predictions and advice related to crop management using an artificial intelligence module, and notify a user terminal with the generated predictions and advice.

### CROSS-REFERENCE TO RELATED APPLICATION

This application is based on and claims priority under 35 USC 119 from Japanese Patent Application No. 2024-144820 filed on Aug. 26, 2024, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional crop management systems often require manual monitoring of soil conditions, moisture levels, and light intensity, resulting in increased labor, delayed responses to crop needs, and a lack of timely and accurate recommendations for optimal crop care. There is a need for an integrated system that can automatically collect and process environmental data, provide predictive analytics using artificial intelligence, and deliver timely advice to users, thereby improving the efficiency and quality of agricultural operations.

### SUMMARY

The present invention provides a system comprising a processor configured to receive measurement data from a sensor device capable of measuring soil condition, water content, and light intensity, store the measurement data in a database, analyze the stored data with an artificial intelligence module to generate predictions and advice related to crop management, and notify a user terminal with the generated predictions and advice. The sensor device may include a soil moisture sensor, a temperature sensor, and a light sensor. The processor may also verify the format and consistency of the received data before storage, thereby enabling reliable and automated support for crop management.

“Sensor device” means a device configured to measure physical parameters of the environment, such as soil condition, water content, and light intensity, and to transmit the acquired data to another system component.

“Soil condition” means the physical or chemical properties of the soil, including but not limited to parameters such as texture, nutrient content, pH, or other measurable attributes relevant to crop growth.

“Water content” means the quantity or proportion of water present in the soil, which is typically measured as soil moisture and is important for determining irrigation needs.

“Light intensity” means the amount of light present in a given area, often measured in lux or similar units, which affects plant photosynthesis and growth.

“Processor” means a hardware or software component capable of executing programmed instructions to perform data processing tasks, including receiving, storing, analyzing, and transmitting information.

“Database” means a structured collection of data stored and managed electronically, enabling efficient retrieval and storage of measurement data from sensor devices.

“Artificial intelligence module” means a program or set of algorithms designed to analyze data, identify patterns, make predictions, and generate advice for crop management based on historical and real-time information.

“Prediction” means a forecast or estimation generated by the artificial intelligence module regarding future conditions or needs of the crops, such as irrigation timing or disease risk.

“Advice” means actionable recommendations or guidelines generated by the artificial intelligence module to assist users in making decisions about crop management.

“User terminal” means an electronic device, such as a smartphone, tablet, or computer, used by a user to receive, display, and interact with notifications, predictions, and advice sent by the system.

“Notify” means to transmit or deliver information, such as predictions and advice, from the processor to the user terminal in a manner that alerts the user.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

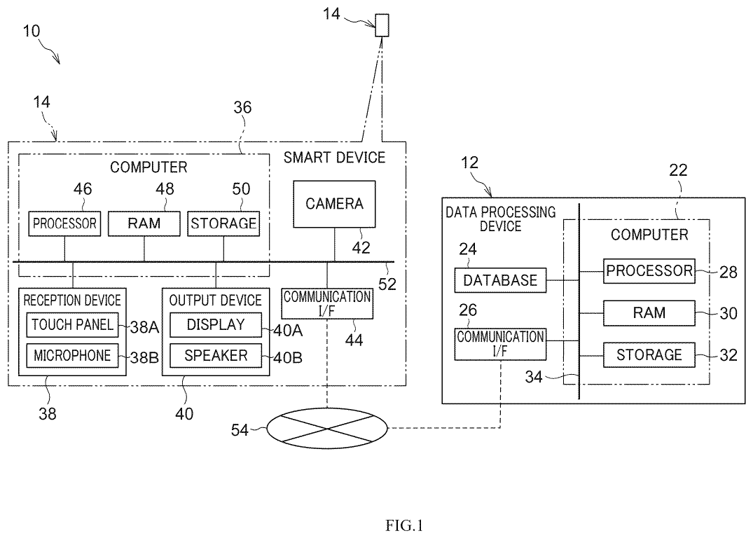

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34.

The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

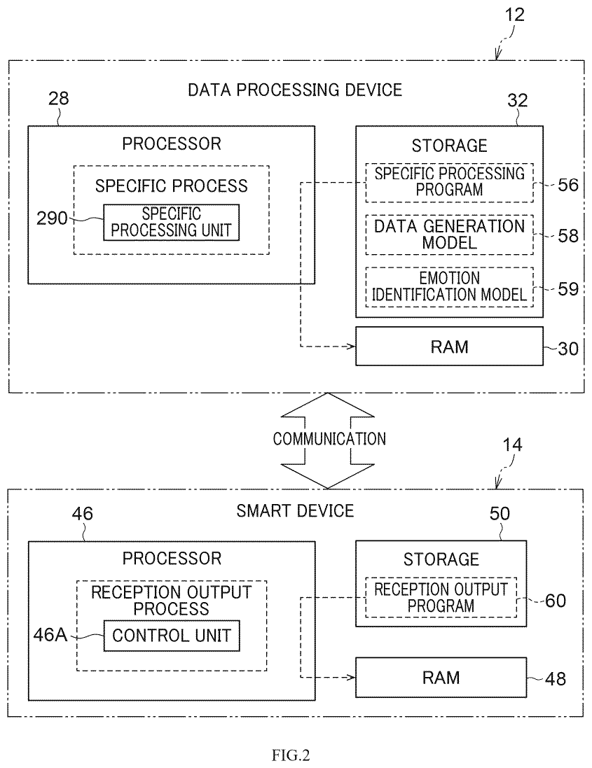

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional agricultural management systems face significant challenges in the continuous and real-time collection of environmental data, comprehensive data analysis, and efficient crop management. In particular, there are difficulties in acquiring consistent and reliable measurements due to missing or erroneous values, and current methods often require manual input to supplement such data. Furthermore, traditional systems lack the ability to utilize advanced predictive analytics for crop management and cannot immediately deliver actionable advice to users, resulting in a decreased efficiency of overall agricultural operations and increased risks to crop yield and quality.

The specific processing by the specific processing unit 290 of the data processing device 12 in Example 1 is realized by the following means.

The present invention provides a server comprising a processor configured to receive environmental measurement data via wireless communication, validate and supplement the data using historical information, store the data in a database, utilize a generative artificial intelligence model in conjunction with a prompt sentence to generate predictions or advice regarding crop management, and notify an information terminal with the generated results. This enables continuous and reliable acquisition, correction, and analysis of environmental data, and allows for timely and effective provision of optimized agricultural management advice to users.

The term “processor” refers to a data processing unit or circuitry capable of executing programmed instructions to perform information processing and control functions within a system.

The term “measurement device” refers to an apparatus equipped with at least one sensor for detecting environmental parameters such as soil moisture, soil condition, temperature, or light intensity, and capable of transmitting corresponding data.

The term “environmental measurement values” refers to physical quantities, such as soil condition, temperature, and light level, that represent the environmental state of a target location at a given time.

The term “wireless communication” refers to a technology or method enabling the transmission and reception of data signals between devices without the use of physical connecting wires, such as Wi-Fi, cellular, or other radio frequency communication.

The term “format and consistency” refers to the conformity of incoming data to predefined data structures and the logical coherence of data values with expected ranges or patterns.

The term “historical data” refers to previously recorded environmental measurement values maintained over time, which can be referenced for validation or supplementation of new data.

The term “database” refers to an organized electronic storage medium used to systematically collect, store, and manage digital data records for efficient retrieval and processing.

The term “generative artificial intelligence model” refers to a computerized algorithm or system that, based on learned patterns from extensive data and user-provided instructions, can generate predictions, advice, or other relevant outputs.

The term “prompt sentence” refers to a textual instruction or query provided to a generative artificial intelligence model for the purpose of specifying the analytical task to be performed.

The term “prediction or advice regarding crop management” refers to forecasted outcomes or recommended actions related to cultivating, irrigating, or protecting crops, based on the analysis of environmental data.

The term “information terminal” refers to an electronic device, such as a smartphone, tablet, or computer, capable of receiving, displaying, or processing notifications and user interaction.

The system comprises a processor included in a server, a measurement device (terminal) installed in the target area such as farmland, and one or more information terminals used by a user. The measurement device is equipped with sensors, such as soil moisture sensors, temperature sensors, and light sensors, to acquire various environmental measurement values. For example, general-purpose environmental sensors may be used for soil moisture, temperature, and light intensity, and a microcontroller may be utilized to aggregate sensor data and support wireless communication protocols such as Wi-Fi or cellular communication.

The measurement device periodically measures environmental parameters, such as soil condition, temperature, and light intensity, and transmits these measurement values wirelessly to the server. The server, functioning as the central processor, receives the measurement data via a network interface, such as a Wi-Fi or cellular module, and validates the format and consistency of the incoming data. As an example, if the server detects missing or abnormal sensor values, it automatically supplements the data by referencing historical data stored in a database. The database may be implemented on a computer-readable storage medium such as a hard disk or SSD, and managed using commonly available database management systems such as a relational database program.

The server stores the validated and supplemented data in the database with appropriate time-stamping. Periodically or on demand, the server extracts stored environmental measurement values and prepares the data for analysis by an artificial intelligence module. In this embodiment, the server generates a prompt sentence, which defines the analytical objective or questions for the generative artificial intelligence model.

For example, the prompt sentence may be:

“Based on the records of soil moisture, temperature, and light intensity for the last seven days, predict the optimal watering timing and amount for the next 48 hours.”

This prompt sentence is provided along with the environmental measurement values to the generative artificial intelligence model, which may be implemented using a machine learning software framework such as TensorFlow or Keras.

The artificial intelligence module then analyzes the input data in accordance with the prompt sentence, and generates predictions or advice regarding crop management, such as irrigation timing and recommended water amount. The server receives the output from the artificial intelligence module, formats the output as a human-readable message, and notifies the information terminal used by the user. Notification may be performed using various communication methods, such as push notification to a mobile application, email, or SMS, depending on the configuration of the information terminal.

The user, upon receiving the prediction or advice on their information terminal-such as a smartphone or tablet—performs the recommended crop management activity in the field. In this way, the system supports real-time and partially automated agricultural management by integrating continuous sensor data collection, intelligent analysis by a generative AI model, and timely user notification.

This embodiment can be realized using general-purpose computer hardware, sensor equipment, and well-known software programs as described above. By employing automatic data validation, supplementation, and intelligent prediction, the system greatly enhances the reliability and efficiency of agricultural operations.

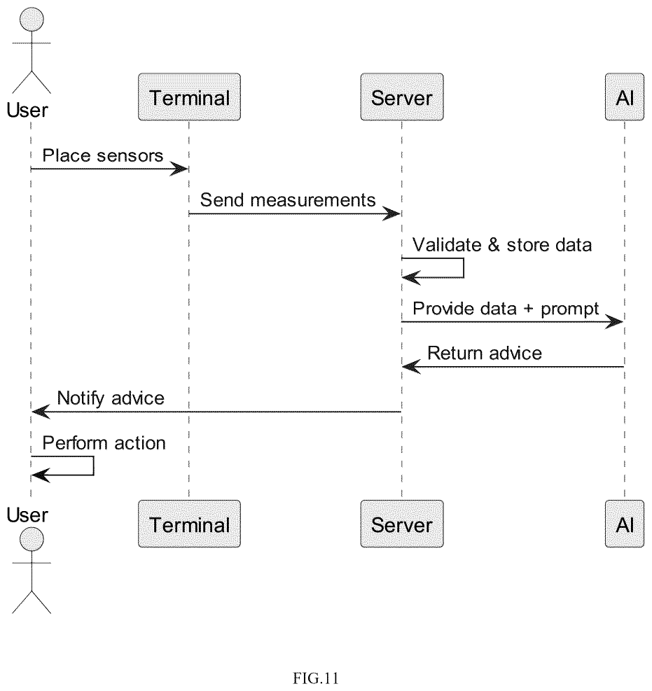

The following describes the processing flow using FIG. 11.

### Step 1:

The terminal measures environmental parameters such as soil moisture, temperature, and light intensity at predetermined intervals.

Input: Environmental conditions in the field (physical quantities such as moisture, temperature, sunlight).

Processing: The terminal uses sensor hardware to collect analog signals, converts them to digital values, and stores the latest readings in its local memory.

Output: Digital measurement data for soil moisture, temperature, and light intensity with timestamp.

Concrete action: The terminal records measurements every three hours, such as “June 12, 9:00 AM—Soil moisture: 27%, Temperature: 23° C., Light: 2100 lux.”

### Step 2:

The terminal transmits measurement data to the server via wireless communication.

Input: Digital measurement data stored in the terminal's memory.

Processing: The terminal formats the data (e.g., JSON), establishes a wireless network connection such as Wi-Fi or cellular, and transmits the data to the server.

Output: Transmitted data packet delivered to the network address of the server.

Concrete action: The terminal sends a JSON object containing the measurement values via HTTP POST request to the server's IP address.

### Step 3:

The server receives and validates the incoming measurement data.

Input: Data packet received from the terminal.

Processing: The server checks the completeness and correctness of the data fields (e.g., presence of all sensor values, valid range). If it detects missing or abnormal values, it references historical records stored in its database to estimate and supplement those values using statistical or rule-based methods.

Output: Validated and supplemented measurement data.

Concrete action: The server recognizes a missing light intensity value, retrieves data from the same time slot on previous days, and fills in the likely value.

### Step 4:

The server stores the supplemented measurement data in the database.

Input: Validated and supplemented measurement data with timestamp.

Processing: The server formats the data as a database record and inserts it into the database using a database management system.

Output: Updated database containing historical environmental measurement records.

Concrete action: The server inserts the latest measurement as a new row in the database table for that field location.

### Step 5:

The server extracts stored measurement data and generates a prompt sentence for the generative AI model.

Input: Database records covering a recent time window (e.g., past seven days).

Processing: The server queries the database to retrieve recent measurement data, formats it appropriately, and creates a prompt sentence specifying the prediction task for the AI model.

Output: Formatted measurement data and a prompt sentence.

Concrete action: The server creates a prompt: “Based on the soil moisture, temperature, and light intensity data from the past seven days, predict optimal watering timing and amount for the next 48 hours.”

### Step 6:

The server analyzes the data using the generative AI model based on the prompt sentence.

Input: Formatted measurement data and prompt sentence.

Processing: The server invokes a generative AI model (e.g., using TensorFlow or

Keras), supplies the data and prompt, and processes the model's output.

Output: Generated prediction or advice regarding crop management.

Concrete action: The AI model returns: “Watering is recommended within the next 20 hours. Suggested amount: 45 liters per crop bed.”

### Step 7:

The server notifies the user's information terminal with the prediction or advice.

Input: AI model output (prediction/advice).

Processing: The server formats the output as a human-readable message and sends it to the user's terminal through a communication method such as a mobile app notification, email, or SMS.

Output: Notification displayed on the user's information terminal.

Concrete action: The user receives a mobile app popup: “Irrigation needed within 20 hours-apply 45 liters per bed.”

### Step 8:

The user views the notification and acts on the provided advice.

Input: Notification containing prediction or advice about crop management.

Processing: The user reads the message, interprets the recommendation, and performs the advised agricultural action in the field.

Output: Implementation of optimized crop management based on system advice.

Concrete action: The user waters the field according to the notification within the recommended period.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In conventional environmental monitoring and management systems, there exist challenges in real-time collection, analysis, and utilization of measurement data for fields such as autonomous driving and agriculture. These systems often lack the capability to flexibly generate accurate predictions and actionable advice based on changing environmental factors such as soil, temperature, and light conditions. Furthermore, they do not adequately account for user conditions and emotional states, resulting in generic notifications that may be inefficient or difficult for users to act upon in a timely and effective manner. There is a need for a system that can provide personalized and contextually adaptive notifications, leveraging advanced data processing and artificial intelligence, in order to improve the safety, efficiency, and usability of such applications in various fields.

The specific processing by the specific processing unit 290 of the data processing device 12 in Application Example 1 is realized by the following means.

The present invention provides a server comprising a processor configured to acquire and verify measurement information from a plurality of sensing devices, preprocess the stored information, perform time-series and statistical analysis using an artificial intelligence inference unit, generate prediction and advice information with a generative artificial intelligence model based on a natural language prompt, notify a user device with the resulting information, estimate the user's emotional state, and dynamically adjust the advice information and notification content based on that estimation. This enables real-time, data-driven, and user-adaptive provision of predictive advice and operational guidance, thereby addressing the needs of environments such as autonomous vehicles and agricultural management with improved relevance, accuracy, and user responsiveness.

The term “sensing device” refers to a component or apparatus configured to measure physical parameters in an environment, such as soil properties, temperature, or light, and to output measurement information in a digital or analog form.

The term “measurement information” refers to data generated by sensing devices that represents values for physical quantities, including but not limited to soil condition, temperature, light level, or other environmental factors.

The term “processor” refers to a hardware-based computational unit or a combination of hardware and software routines that is capable of executing instructions for processing data, controlling devices, and managing workflow in the system.

The term “storage medium” refers to any digital or physical device that is capable of recording and retaining data, including but not limited to memory chips, hard drives, solid-state drives, and database servers.

The term “preprocess” refers to operations performed on raw measurement information prior to further analysis, such as filtering abnormal values, supplementing missing data, and aggregating the values over defined time intervals.

The term “artificial intelligence inference unit” refers to a computer-implemented module or engine configured to perform analytical or predictive processing of data using machine learning models, statistical algorithms, or generative models.

The term “generative artificial intelligence model” refers to a software-based system, such as a trained language model, capable of producing human-readable advice, predictions, or recommendations from structured or unstructured data input, including natural language prompt sentences.

The term “natural language prompt” refers to an input sentence or query, composed in human natural language, which instructs or guides the generative artificial intelligence model on the type of prediction or advice to generate.

The term “user display device” refers to any type of electronic hardware through which a user may receive notifications or reports from the system, such as a smartphone, tablet, computer, or vehicle display module.

The term “communication unit” refers to a hardware or software system enabling the transfer of data, advice, or notifications between the processor and a user display device via wired or wireless communication protocols.

The term “biometric information” refers to data about physical or behavioral characteristics of a user, such as facial expressions, voice, heartbeat, or operating patterns, that can be used to estimate emotional state.

The term “emotion recognition unit” refers to a software or hardware component, or a combination thereof, that identifies or estimates a user's emotional state based on biometric information or operation history.

The term “customization unit” refers to a processing module configured to change or adapt advice information and notification content according to the results of emotion estimation, in order to present information more effectively to the user.

### Embodiment for Implementing the Invention

The present invention may be implemented by utilizing a combination of general-purpose computational hardware, sensors, data communication components, and software modules designed for measurement, storage, analysis, prediction, and notification processes. The system consists of one or more servers equipped with processors, storage media, and communication units; a plurality of sensing devices for measuring environmental parameters; and one or more user display devices, such as smartphones or tablets, capable of receiving and displaying information.

The user installs a plurality of sensing devices at locations relevant to the environmental monitoring objective. The sensing devices may measure various physical quantities, such as soil properties (including moisture content and temperature) and ambient light intensity. Hardware such as microcontroller boards (for example, single-board computers or generic sensor modules) may be utilized for this purpose. The sensing devices communicate measurement information to the server wirelessly, using standard protocols such as Wi-Fi, mobile communication, or low-power wide-area networks.

The server receives the measurement information from each sensing device. The server verifies the format and integrity of incoming data, and stores the data in a storage medium such as a database (for example, a relational database like PostgreSQL or a document-based database like MongoDB). The server then preprocesses the stored data—this may be performed using general-purpose programming languages and data processing libraries, such as Python with the pandas library. In preprocessing, the server can remove abnormal values, fill missing data through logical inference, and aggregate values by average or sum over specified time windows.

The server is equipped with an artificial intelligence inference unit, which may be implemented in software using a machine learning framework such as TensorFlow, PyTorch, or scikit-learn. The artificial intelligence inference unit performs time-series analysis and statistical modeling of the preprocessed measurement information, generating predictions and advice information relevant to the target application, such as anticipated risk of slippery surfaces or recommendations for irrigation timing.

A generative artificial intelligence model, such as a state-of-the-art language model, is utilized for producing user-friendly advice. The model receives a natural language prompt constructed by the server, for example:

“Based on the past seven days' soil moisture, temperature, and light readings, predict the risk of surface slipperiness for autonomous vehicles over the next 48 hours and provide advice for the operator.”

or

“Sensor readings: soil moisture=30%, temperature=15° C., light=800 lux. Predict if the crops will require watering or pest control in the next 48 hours and generate advice for the user. Adjust the tone to be friendly if the user is detected as stressed.”

The generated advice and prediction information are sent by the server to the user display device using a communication unit, utilizing push notifications, email, SMS, or a custom mobile application.

The user display device, such as a smartphone, receives the notification and displays it to the user in an easily understandable format. In some embodiments, the terminal may also acquire biometric data, such as facial expressions, voice, or behavioral patterns (for example, touch input or application usage), and transmit these to the server for emotion recognition.

The server analyzes biometric or interaction data using an emotion recognition unit implemented by a software library or framework for sentiment analysis, such as natural language processing toolkits or customized machine learning models. If the user's emotional state is estimated to be stressed or busy, the server utilizes a customization unit to adapt the advice to be shorter or more encouraging in content and tone, thereby enhancing usability and responsiveness.

For example, if prediction information indicates an increased risk of slippery conditions in the early morning and emotion analysis detects user fatigue, the advice may be:
“Quick tip: Please drive with extra caution tomorrow morning!”

By combining accurate environmental sensing, advanced data analysis, flexible advice generation using generative AI models, and user-adaptive notification features, the invention ensures that users can reliably receive relevant, timely, and actionable predictions and recommendations for target management scenarios, such as autonomous vehicle operation or agricultural field management.

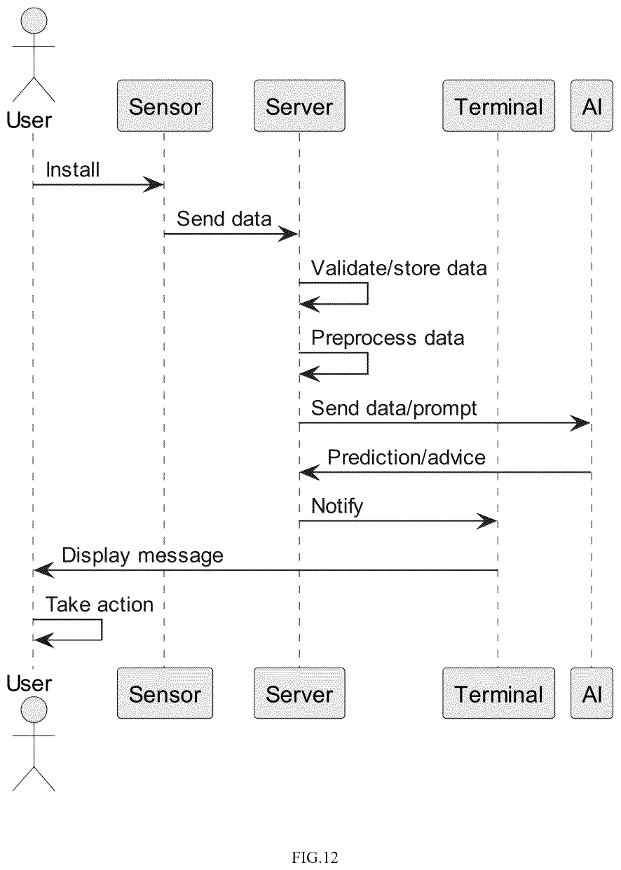

The following describes the processing flow using FIG. 12.

### Step 1:

User installs multiple sensing devices in the target environment. The input is the physical deployment of sensors such as soil moisture, temperature, and light sensors. The output is a set of operational sensing devices ready to collect environmental data. The specific action involves attaching or inserting the sensors into the ground or mounting them in positions where accurate measurement can be obtained.

### Step 2:

Sensing devices acquire environmental measurements at regular intervals. The input is environmental physical phenomena (e.g., soil moisture, ambient temperature, and light levels). The output is digital measurement data, typically formatted with a timestamp, sensor ID, and measured values. The devices perform analog-to-digital conversion and package the data for transmission.

### Step 3:

Sensing devices wirelessly transmit collected measurement data to the server. The input is the formatted measurement data from each device. The output is data packets received by the server via a wireless protocol such as Wi-Fi or cellular communication. The devices execute network communication routines to send the data to a designated network address of the server.

### Step 4:

Server receives measurement data and verifies its format and integrity. The input is the incoming data packets from the sensing devices. The output is validated data records. The server checks completeness, correct data types, and valid value ranges; missing or abnormal values are logged for correction.

### Step 5:

Server stores the verified measurement data in a storage medium such as a database. The input is validated sensor data. The output is a structured record set in the database, organized by sensor ID and timestamp. The server runs database insertion operations to achieve persistent storage.

### Step 6:

Server preprocesses the stored data. The input is the historical sensor data from the database. The output is a cleaned and aggregated dataset. The server filters out abnormal data points, fills gaps using prior or average values, and aggregates measurements over specified intervals (for example, computing hourly averages). This is performed using data processing libraries such as pandas in Python.

### Step 7:

Server compiles a dataset and generates a prompt sentence for generative AI analysis. The input is the preprocessed and aggregated data. The output is a formatted dataset and a natural language prompt describing the desired analysis (e.g., risk prediction or advice generation). The server prepares input according to the requirements of the AI model or API.

### Step 8:

Server sends the dataset and prompt sentence to the generative AI model for inference. The input is the formatted dataset and prompt. The output is generated prediction and advice information in natural language. The AI model analyzes trends and applies learned relationships to create actionable recommendations.

### Step 9:

Server receives the response from the generative AI model and formats it for user communication. The input is the generated prediction and advice. The output is a notification message or report intended for the user. The server may further adapt the content for clarity or local language.

### Step 10:

Server analyzes user input, biometric data, or operation logs from the user terminal to estimate the user's emotional state. The input is biometric or behavioral data (such as facial expression, voice, or interaction pattern) from the terminal. The output is an estimated emotion state (such as “relaxed,” “stressed,” or “indifferent”). The server processes this data using an emotion recognition algorithm or model.

### Step 11:

Server customizes the message content based on the user's estimated emotional state. The input is the initial advice message and the emotion estimation result. The output is a modified notification tailored to increase user comprehension and engagement. For example, if the user is stressed, the advice is made brief and encouraging.

### Step 12:

Server transmits the customized message to the user terminal. The input is the tailored notification or report. The output is a push notification, email, or SMS received by the user's device. The server uses communication APIs or notification services to deliver the message.

### Step 13:

Terminal displays the notification to the user. The input is the received message from the server. The output is a user-readable display of advice and predictions. The terminal runs the interface app or uses built-in notification features to ensure the user easily receives and understands the provided information.

### Step 14:

User checks the notification and takes the recommended action. The input is the displayed advice and prediction results. The output is the user's operational response in the real world, such as adjusting irrigation, changing travel plans, or modifying machinery operation. The user reads, interprets, and acts based on the advice.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional environmental management systems for agricultural fields lack robust mechanisms to supplement incomplete or inconsistent sensor data, resulting in reduced accuracy of predictive analytics and guidance. Moreover, existing systems do not take into account the emotional state of users when generating notifications, which may lead to ineffective communication and lower user satisfaction. There is a need for a system that ensures higher data reliability and provides personalized notifications adapted to the user's emotional condition, thereby improving both predictive precision and usability in environmental or crop management.

The specific processing by the specific processing unit 290 of the data processing device 12 in Example 2 is realized by the following means.

The present invention provides a server comprising a processor configured to receive measurement data from sensor units via wireless communication, store the measurement data with context and unique identifiers, validate and supplement the data using statistical and mathematical methods, export the processed data in a time-series format to an external processing apparatus, and, with the help of machine learning, generate recommendation information. The server is further configured to analyze user emotion through image or audio data, optimize notification content based on the emotional state, and transmit such personalized notifications to a terminal device. This enables highly accurate prediction and guidance for environmental management, as well as emotionally adaptive notifications that improve user engagement and operational efficiency.

The term “sensor unit” refers to a measurement device that acquires environmental information such as soil moisture, temperature, and light intensity, and is capable of transmitting the collected data via wireless communication.

The term “measurement data” refers to digital information representing physical parameters of an environment, including but not limited to soil moisture levels, ambient temperature, and light levels, as captured by a sensor unit.

The term “processor” refers to an electronic circuit or computer device configured to execute programmed operations such as receiving, analyzing, validating, and supplementing measurement data.

The term “storage device” refers to an electronic component or system for storing digital data, including measurement data, context information, and unique identifiers, typically realized by memory devices such as hard drives or semiconductor memory. The term “context information” refers to supplementary data that describes the circumstances surrounding each measurement, including temporal data, location data, or sensor identification data.

The term “unique identifier” refers to a distinctive code or data element that is associated with each sensor unit or measurement record to enable individual recognition and traceability.

The term “statistical or mathematical methods” refers to analytical procedures or algorithms, such as linear interpolation or time-series modeling, used to estimate or complete missing or anomalous measurement data.

The term “external processing apparatus” refers to a computational device or server that receives, stores, and analyzes measurement data provided by another system, often using advanced methods such as machine learning or artificial intelligence.

The term “external data description format” refers to a structured digital representation, such as CSV or JSON, used to export and communicate measurement data from one system to another.

The term “machine learning computation device” refers to a computing resource equipped with software and hardware capable of executing machine learning algorithms to analyze environmental data and generate predictions or recommendations.

The term “recommendation information” refers to suggested actions or guidance provided to a user, such as operational strategies or instructions based on the analysis of measurement data.

The term “image information” refers to digital data derived from visual input, such as photographs or video frames, particularly of the user's face or expression.

The term “audio information” refers to digital data constituted by recorded sound, especially the user's voice or other expressions emitted during system interaction.

The term “emotion estimation processing apparatus” refers to a computational system or software module configured to analyze image information and audio information for the purpose of determining a user's emotional state.

The term “notification content” refers to the messages or alerts generated by the system to inform the user, including guidance, warnings, or recommendations.

The term “terminal device” refers to an electronic user interface device, such as a smartphone, tablet, or computer, that receives and presents notification content to the user.

The term “generative artificial intelligence model” refers to a machine learning-based algorithm or framework that produces predictions, recommendations, or analyses by generating output based on input data and user-provided prompt sentences.

The term “prompt sentence” refers to a text-based input provided to the generative artificial intelligence model to specify the type of prediction or analysis to be performed.

The embodiments for carrying out the invention will be described below based on the technical scope of the appended claims.

This invention can be implemented by configuring a system comprising at least a sensor unit, a server, and a terminal device, in accordance with the following structure and hardware/software components.

The user installs multiple sensor units within an environment such as an agricultural field. Each sensor unit is equipped with measurement elements capable of acquiring environmental data, such as soil moisture, temperature, and light intensity, and is furnished with a wireless communication module, for example using the ZigBee or LoRa protocol. The sensor unit assigns a unique identifier and transmits measurement data in a standardized data format.

The server is configured with a processor and a storage device. The server receives the measurement data from the sensor units via its network gateway. Upon receiving the data, the server uses software components such as a Python script or equivalent middleware to check the completeness and validity of incoming data. The data, including context information (like timestamps, location, and sensor IDs), is stored in a MySQL or similar relational database system.

When the server detects missing or abnormal data, the server applies statistical or mathematical methods, such as linear interpolation or a time-series model implemented with libraries like pandas or scikit-learn, to impute missing values and supplement irregular data entries. This ensures the integrity of the stored dataset.

The server regularly exports time-series measurement data, for example in CSV or JSON format, for further analysis by an external processing apparatus. This external processing apparatus may be integrated or remote and is equipped with a computation device capable of executing machine learning algorithms. In one implementation, a generative artificial intelligence model, built on the TensorFlow framework, is used to process environmental data and generate predictive analytics and recommendation information regarding the state of the target organism, such as crop growth status or irrigation needs. The user interacts with the terminal device, such as a smartphone or tablet. When the user accesses status reports, the terminal device acquires image information using the built-in camera and audio information using the microphone, with the user's permission. The terminal transmits these data securely to the server. The server executes an emotion estimation processing apparatus, using software such as DeepFace for image analysis and OpenSMILE for audio analysis, to estimate the emotional state of the user from the received image and audio data.

Based on the analysis, the server creates personalized notification content. If the emotion estimation indicates that the user is busy or stressed, the server optimizes the notification to be concise or encouraging. The server sends the final notification to the terminal device via secure channels, such as Firebase Cloud Messaging for application push notifications, email, or SMS. The terminal immediately presents the tailored message to the user.

A concrete example of the system in use is as follows. The user places several sensor units throughout an agricultural site. The sensors collect measurements every three hours and transmit the data wirelessly. The server stores the data in a MySQL database, applies interpolation if any measurements are missing, and at a configured time each day, exports the most recent data for AI processing. The TensorFlow-based generative AI model analyzes the previous thirty days of history and today's data and predicts that the crops will require watering within twenty-four hours. When the user checks this notification on their mobile device, the device captures an image and audio sample. If the emotion estimation reveals the user is under stress, the notification presented will be brief and direct, for instance: “Please water your crops in the next 24 hours.”

An example prompt sentence that can be input to the generative AI model is as follows:

“Analyze the sensor data and predict environmental changes over the next 48 hours. Generate recommendations regarding the need for watering and specific advice for the user.” By using this system structure and the described processes, the invention allows for robust collection, validation, supplementation, analysis, and user-adaptive presentation of environmental management information.

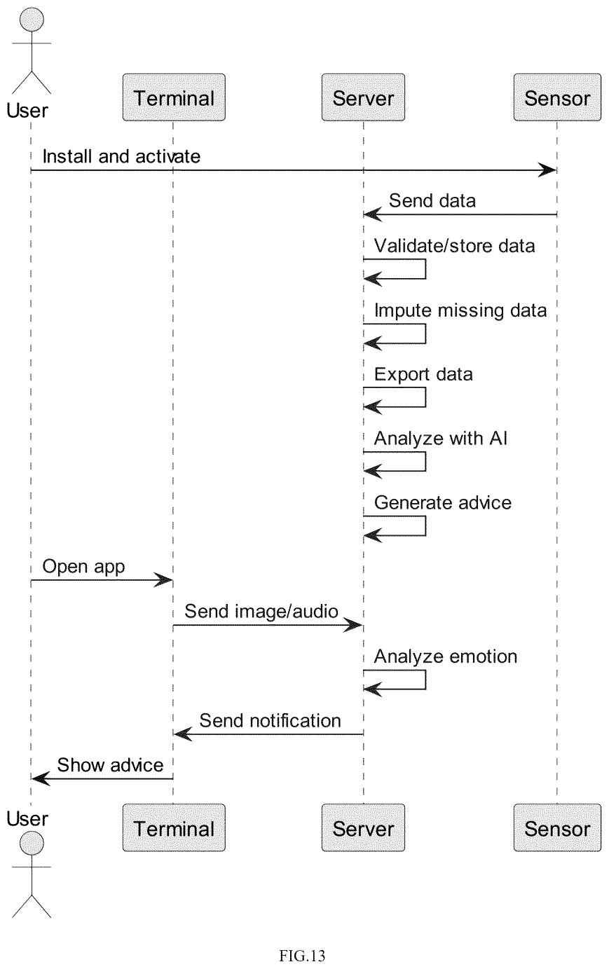

The following describes the processing flow using FIG. 13.

### Step 1:

User installs multiple sensor units in the environment, such as an agricultural field, and powers on each device. Each sensor unit starts measuring environmental parameters such as soil moisture, temperature, and light intensity at configurable intervals (for example, every three hours).
Input: None (physical installation by user)
Output: Environmental measurement data generated by each sensor unit.

### Step 2:

Sensor units aggregate the measured environmental data and use a wireless communication module to transmit the measurement data (including unique identifier and timestamps) to the server.
Input: Environmental measurement data from sensors
Output: Wireless data packets containing measurement data, transmitted to the server.

### Step 3:

Server receives incoming data packets from each sensor unit via the network gateway. Server parses the packets to extract values for soil moisture, temperature, light intensity, timestamp, and sensor ID. Server checks the format, completeness, and validity of each data field using a parsing routine implemented in Python or similar language.
Input: Data packets received from sensor units
Operation: Data parsing, field validation, and completeness checking
Output: Structured and validated measurement data.

### Step 4:

Server stores the structured and validated measurement data into a relational database (for example, MySQL), along with context information such as timestamp and sensor ID.
Input: Validated measurement data from Step 3
Operation: Database write operation with context information
Output: Persistent storage of measurement data entries.

### Step 5:

Server examines each new database entry for missing, inconsistent, or abnormal values. Server applies data supplementation algorithms, such as linear interpolation or time-series modeling using mathematical libraries, to impute missing or abnormal data points.
Input: Database records with raw measurement data
Operation: Statistical or mathematical data imputation
Output: Database records with complete and consistent data.

### Step 6:

Server periodically exports time-series measurement data from the database in a standardized external file format (e.g., CSV or JSON) for further processing.
Input: Supplemented and complete measurement records from the database
Operation: Data export in external data format
Output: Time-series data file (CSV or JSON) for analysis.

### Step 7:

Server provides the exported time-series measurement data to an external processing apparatus or invokes an internal generative AI model, for example, using TensorFlow. Server may also input a prompt sentence specifying the required analysis, such as:

“Analyze the sensor data and predict environmental changes over the next 48 hours. Generate recommendations regarding the need for watering and specific advice for the user.”
Input: Time-series measurement data; prompt sentence
Operation: Execution of generative AI model; data analysis and prediction
Output: Predictive analytics and recommendation information (e.g., JSON with advice).

### Step 8:

Server receives the output from the generative AI model, which includes predictive analytics and recommendation information. Server logs and stores this information for notification processing.
Input: Recommendation information from the generative AI model
Operation: Data logging and storage of recommendations
Output: Recommendation data prepared for user notification.

### Step 9:

User accesses the terminal device, such as a smartphone, and opens the dedicated application to check the current status report. Terminal device, upon user interaction, activates the camera and microphone to capture a brief sample of the user's facial image and voice.
Input: User interaction (opening report screen)
Operation: Collection of image and audio samples by the terminal device
Output: Image and audio files containing user emotional cues.

### Step 10:

Terminal device securely transmits the captured image and audio information to the server via an encrypted communication channel.
Input: Image and audio files from the terminal device
Operation: Secure data transmission
Output: User image and audio data received by the server.

### Step 11:

Server invokes an emotion estimation processing apparatus implemented with image and audio analysis tools (such as DeepFace and OpenSMILE) to process the received user data. Server analyzes the user's facial expression and voice to estimate their emotional state.
Input: User image and audio data
Operation: Emotion recognition by processing apparatus
Output: Determined user emotional state (e.g., stressed, busy, neutral, positive).

### Step 12:

Server personalizes the notification content based on the emotional state of the user. If the user is recognized as busy or stressed, server modifies the message to be more concise or supportive. Server combines the AI recommendations with the personalized message content.
Input: Recommendation information and user emotional state
Operation: Notification content optimization and composition
Output: Optimized notification ready for transmission.

### Step 13:

Server transmits the personalized notification content to the terminal device using an appropriate delivery method, such as push notification via Firebase, email, or SMS.
Input: Optimized notification content
Operation: Message delivery via selected channel
Output: Notification received on terminal device.

### Step 14:

Terminal device immediately presents the notification content to the user in the application's user interface. User reads the notification and is able to take action based on the received recommendations (for example, initiate irrigation).
Input: Notification content delivered to terminal device
Operation: Display of notification to user
Output: User awareness of guidance; user may execute recommended action.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional environmental monitoring and management systems are limited in their ability to promptly and accurately predict risks such as abnormal temperature, moisture, or other hazardous conditions. Furthermore, these conventional systems do not consider the emotional state of the user when delivering notifications, which may result in ineffective communication, excessive stress, or inappropriate responses in emergency situations. Therefore, there is a need for a system that can not only analyze sensor data for risk management but also adjust notification content in accordance with the user's current emotional state, thereby improving safety and user experience.

The specific processing by the specific processing unit 290 of the data processing device 12 in Application Example 2 is realized by the following means.

The present invention provides a server comprising a processor configured to acquire measurement information from an information acquisition device, validate and supplement such measurement information, analyze the information using a machine learning technique or a generative information processing model, generate prediction and advice information related to environment management, adjust the prediction and advice information based on a user's emotional state, and notify the adjusted information to a user terminal device. This enables accurate and timely risk prediction as well as communication that is adaptive to the user's emotional context, thereby enhancing both safety and user satisfaction.

The term “processor” refers to a hardware or software component configured to perform data processing, control operations, and execute programmed instructions within the system.

The term “information acquisition device” refers to one or more devices or modules capable of measuring and obtaining data related to physical or environmental conditions, including but not limited to sensors.

The term “measurement information” refers to the data acquired by the information acquisition device that represents one or more physical or environmental parameters such as temperature, humidity, or illumination.

The term “storage device” refers to a hardware or software component configured to store measurement information received from the information acquisition device in a retrievable manner.

The term “validation” refers to the process of verifying the format, integrity, and reliability of the measurement information before further analysis or use.

The term “analysis” refers to the processing of measurement information using techniques such as statistical analysis, machine learning, or artificial intelligence models to derive assessments, predictions, or actionable insights.

The term “machine learning technique” refers to an algorithmic method that enables analysis or prediction based on patterns learned from historical data.

The term “generative information processing model” refers to an artificial intelligence model configured to generate outputs, such as text or predictions, based on input data, and includes models based on generative algorithms.

The term “prediction information” refers to results derived from analyzing measurement information to forecast potential risks, abnormalities, or future conditions.

The term “advice information” refers to suggestions or recommendations for actions, generated in response to prediction information, to guide users in managing and responding to environmental conditions.

The term “notification adjustment unit” refers to a component configured to modify the content or presentation of notifications based on factors such as the emotional state of the user.

The term “emotional state” refers to the current mental or affective condition of the user, which can be recognized or inferred from user behaviors, expressions, or interactions.

The term “user terminal device” refers to any electronic device, such as a smartphone or computer, utilized by the user to receive notifications or interact with the system.

The system includes a server comprising a processor, one or more information acquisition devices (e.g., environmental sensors), storage devices, a machine learning module or generative information processing model, a notification adjustment unit, and user terminal devices such as smartphones or computers.

The information acquisition device consists of physical sensors capable of measuring environmental parameters including, but not limited to, temperature, humidity, and illumination. These sensors may be implemented using commercially available products, such as capacitive soil moisture sensors, temperature sensors (e.g., DHT22), and light sensors (e.g., LDRs), which are connected to communication modules (e.g., Wi-Fi, Zigbee, or Bluetooth modules). The sensors periodically acquire measurement information and wirelessly transmit the data to the server.

The server receives measurement information from the information acquisition devices using a software interface such as a REST API built with Python Flask or Node.js Express. The server parses and stores the received data in a storage device, which may be configured as a relational or non-relational database, such as MySQL, PostgreSQL, or MongoDB. The server validates the format and reliability of the measurement information. If defective or missing information is detected, the server supplements such information by interpolation or referencing historical data, utilizing software libraries such as pandas for data processing.

For analysis, the server uses a machine learning module or a generative information processing model, such as those implemented with frameworks like TensorFlow, PyTorch, or Scikit-learn. These modules analyze the validated sensor data to detect anomalies and generate prediction information concerning environmental risk. The processor further generates advice information, providing users with appropriate recommendations tailored to the detected risks.

When preparing notifications, the server employs a notification adjustment unit, which customizes the prediction and advice information according to the inferred emotional state of the user. The user terminal device, such as a smartphone, captures information relevant to the emotional state, for example, facial features using a camera or vocal features using a microphone. The captured data is transmitted to the server, where emotion recognition is performed using software such as OpenCV for facial expression analysis or Google Speech-to-Text for voice analysis. The server infers the user's emotional state and utilizes this information to adjust the urgency, length, and tone of the notification. The adjustment itself may be performed using a generative AI model, such as a large language model, in response to a specific prompt sentence describing the desired notification style.

For example, if the user is detected to be stressed, the server may use a prompt such as:

“Shorten the notification message for a user who is in a stressful state. If there is a high security risk, generate a brief and urgent notification message urging immediate action.” The server then sends the adapted notification to the user terminal device via push notification, email, or SMS, using services such as Firebase Cloud Messaging, SMTP servers, or SMS gateways.

The user receives the notification on the terminal device, reviews the prediction and advice information, and can take appropriate action, such as checking real-time environmental status, activating alarms, or providing feedback about the notification. This feedback may be used by the server to further train and improve the notification and emotion recognition models.

Through this configuration and process, the system enables real-time monitoring and management of environmental risks and delivers adaptive, user-oriented communication that enhances both safety and user experience.

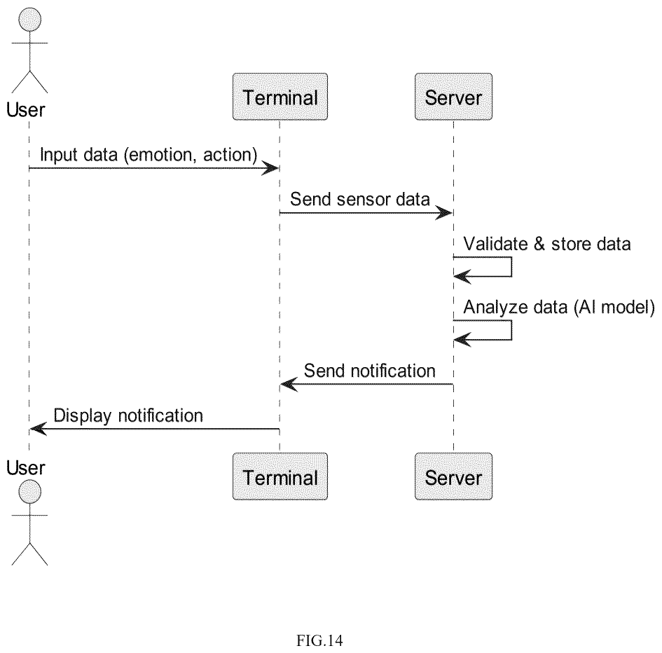

The following describes the processing flow using FIG. 14.

### Step 1:

The terminal (sensor device) measures environmental parameters such as temperature, humidity, and illumination using built-in sensors. As input, the terminal obtains physical data from the environment. The terminal processes this raw data, formats it into structured measurement information (such as a JSON object), and then transmits it wirelessly to the server as output.

### Step 2:

The server receives the structured measurement information from the terminal via a network endpoint. As input, the server accepts the transmitted data packages. The server parses the data, checks each value for presence, and stores the validated records in a storage device such as a database. The output is a set of cleaned and structured measurement data stored for later use.

### Step 3:

The server performs validation and supplementation of the stored measurement information. As input, the server retrieves records from the storage device. The server analyzes the data format and checks for missing or abnormal values. If incomplete data is detected, the server uses historical records or interpolation algorithms to supplement the missing values. The output is corrected and verified measurement information.

### Step 4:

The server analyzes the validated measurement information by using a machine learning module or generative AI model. As input, the server uses multiple recent and historical records from the database. The server runs inference operations-such as anomaly detection or environmental predictions-on this data. The output is prediction information and advice information that identify potential environmental risks and recommend actions.

### Step 5:

The user terminal captures user-related data, such as facial expressions via a camera or vocal tone via a microphone, during the notification review. As input, the terminal collects audio-visual signals from the user. The terminal converts and transmits feature data representative of emotional cues to the server. The output is a transmission of emotional state data for further analysis.

### Step 6:

The server receives the emotional state data from the terminal. As input, the server obtains user emotion features and previously generated prediction and advice information. The server uses emotion recognition algorithms to infer the user's emotional state based on received cues, and then utilizes a generative AI model to adapt the tone, urgency, and content of the notification. The output is an adjusted and customized notification message.

### Step 7:

The server transmits the customized notification message to the user terminal using push notification services, email systems, or SMS gateways. As input, the server uses the adjusted notification content and target terminal address. The output is a delivered notification on the user's selected communication device.

### Step 8:

The user receives the notification on the terminal, reviews the prediction and advice information, and decides on an appropriate action, such as checking live environmental data or activating emergency responses. As input, the user gets the notification message and any related links or control options. The user responds accordingly, and any feedback or actions taken can be optionally sent back from the terminal to the server for logging or learning purposes. The output is user action and/or feedback data, enabling further optimization of the system.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

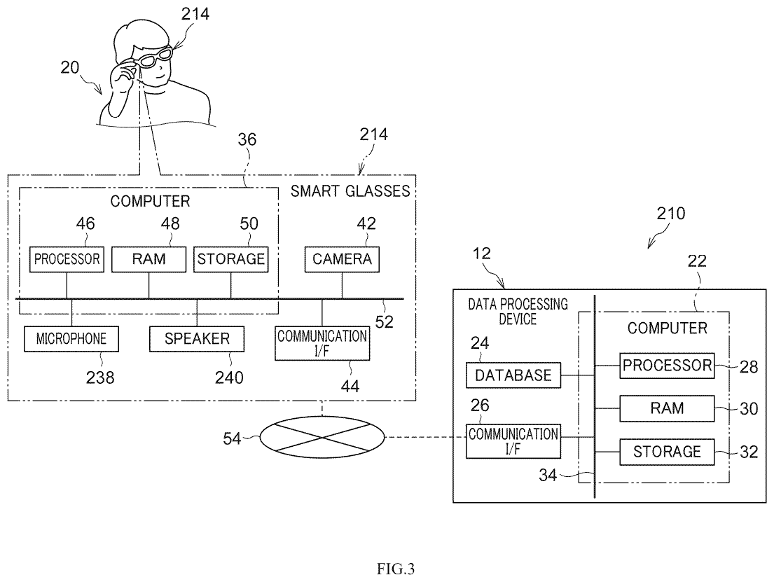

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34.

The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

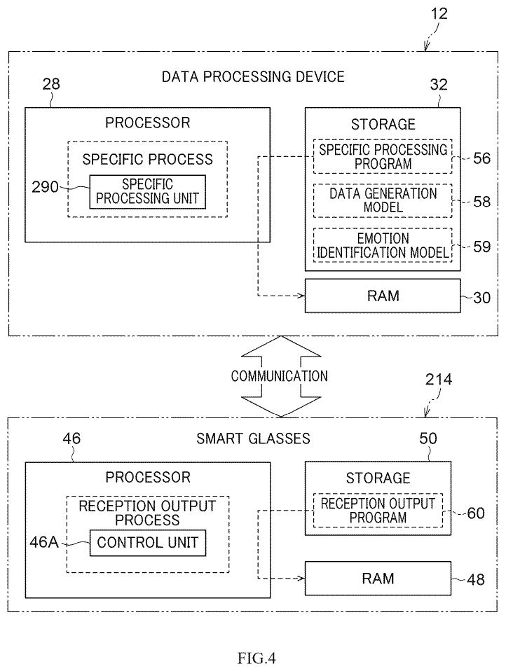

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

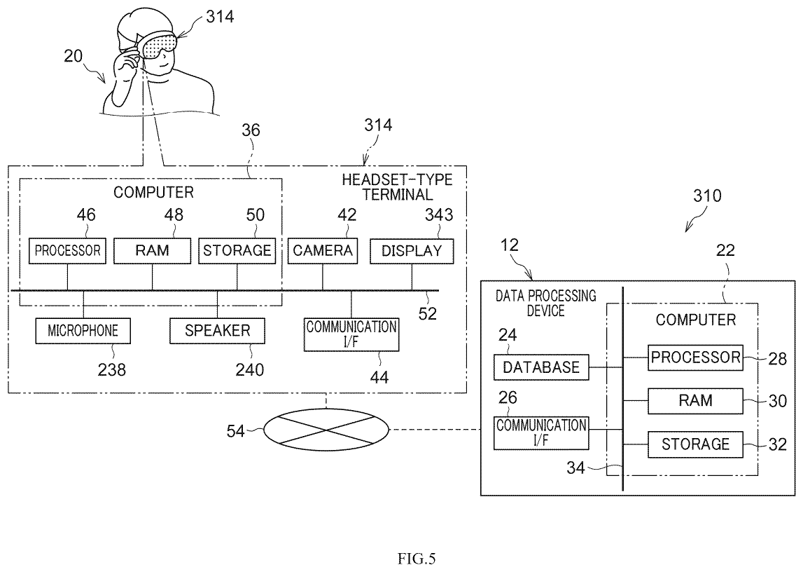

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34.

The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

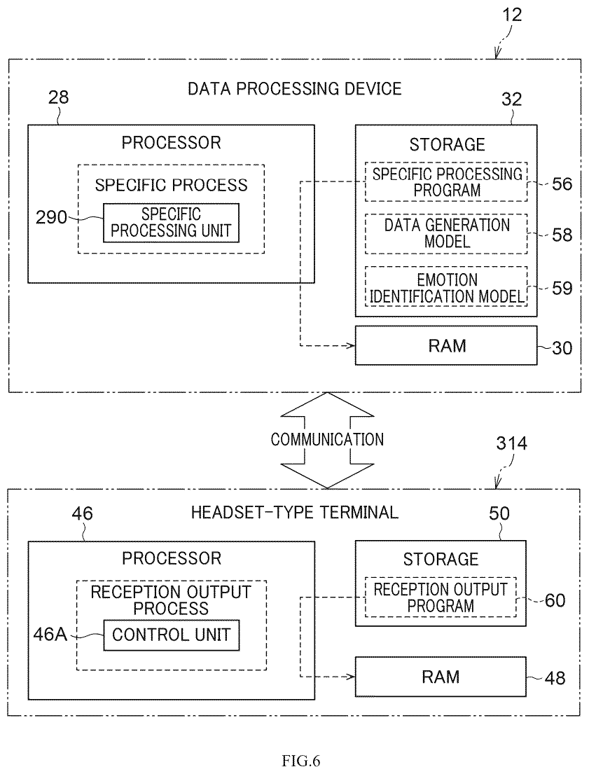

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

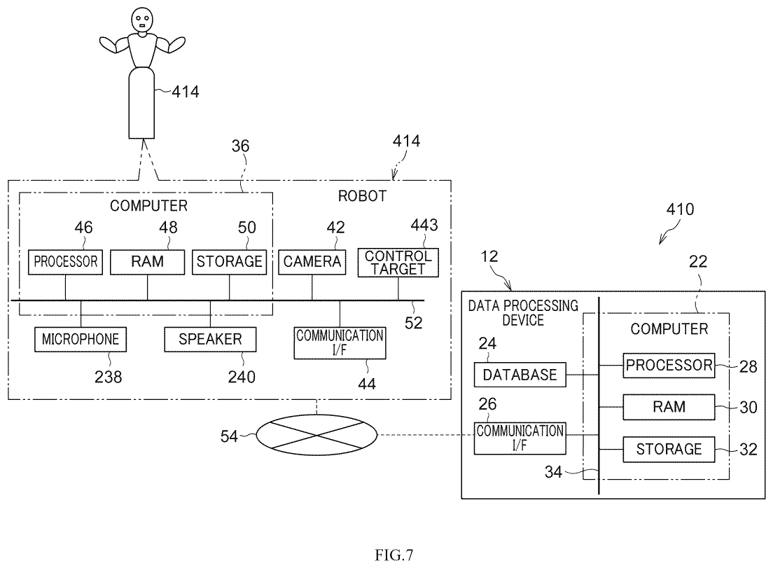

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34.

The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

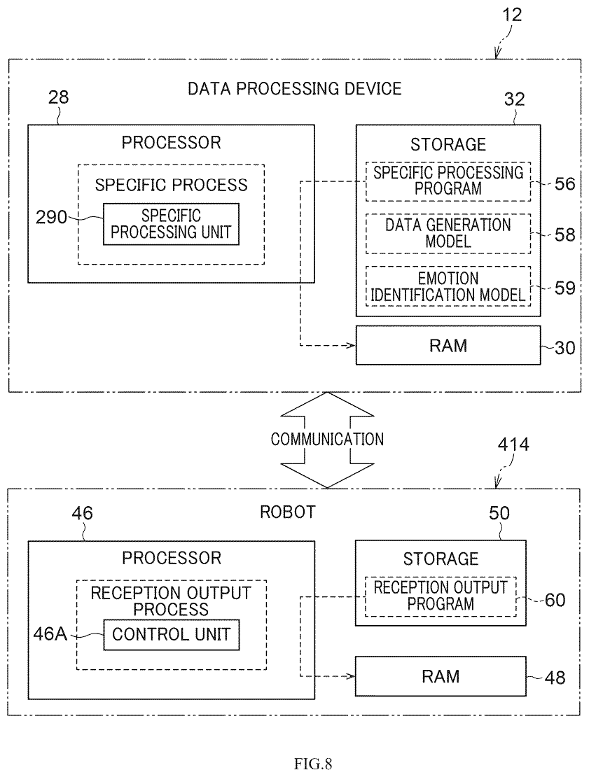

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56.

The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

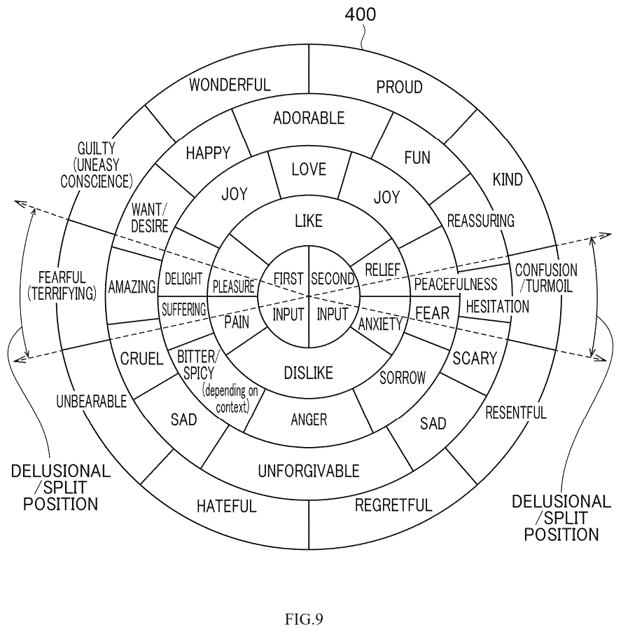

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

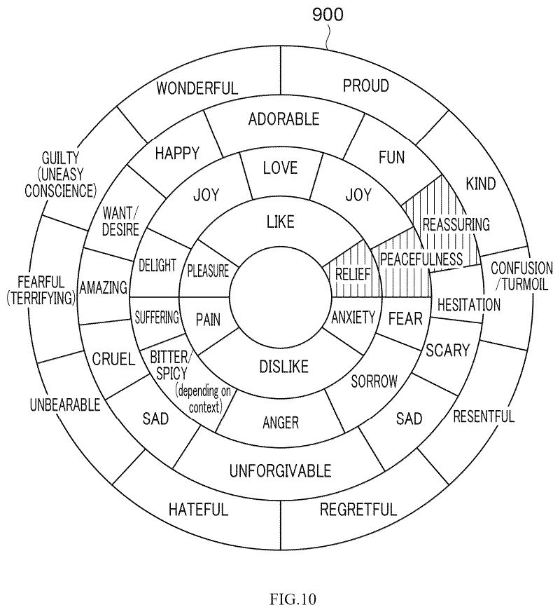

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
receive data from a measurement device that acquires environmental measurement values via wireless communication,
verify the format and consistency of the received data, supplement any abnormal or missing values based on historical data, and store the data in a database,
extract the stored data and provide a prompt sentence to a generative artificial intelligence model to generate a prediction or advice regarding crop management, and
notify an information terminal of the generated prediction or advice.

### (Supplementary 2)

The system according to supplementary 1,
wherein the processor is configured to
enable the measurement device to measure environmental information including soil condition, temperature, and light environment.

### (Supplementary 3)

The system according to supplementary 1,
wherein the processor is configured to
cause the generative artificial intelligence model to output a recommended crop management action based on the input environmental measurement values and the prompt sentence.

### Application Example 1

### (Supplementary 1)

A system comprising a processor,
wherein the processor is configured to
acquire measurement information from a plurality of sensing devices that measure physical quantities and transmit said measurement information,
receive the measurement information from the sensing devices, verify the format and integrity of the information, and store the information in a storage medium,
preprocess the stored measurement information by excluding or supplementing abnormal values and aggregating data over a time period,
execute time-series analysis and statistical processing of the preprocessed measurement information to generate prediction information and advice information regarding environmental conditions or target management using an artificial intelligence inference unit,
utilize a generative artificial intelligence model receiving a natural language prompt as input to generate said advice information,
notify a user display device of said prediction information and advice information via a communication unit,
estimate a user emotional state based on biometric information or operation history received from the user display device using an emotion recognition unit, and
adjust the advice information and notification content according to the estimated user emotional state by a customization unit.

### (Supplementary 2)

The system according to supplementary 1,
wherein the processor is configured to
cause the plurality of sensing devices to comprise multiple types of elements capable of measuring physical quantities related to soil, temperature, and light amount.

### (Supplementary 3)

The system according to supplementary 1,
wherein the processor is configured to
dynamically select the content and expressions of the prediction information and advice information in response to a prompt sentence provided to the artificial intelligence inference unit.

### Example 2

### (Supplementary 1)

A system comprising a processor,
wherein the processor is configured to
receive environmental condition measurement data transmitted by a sensor unit via wireless communication,
store the measurement data together with context information and unique identifiers in a storage device,
check the completeness and validity of the received data, and supplement incomplete or missing data using statistical or mathematical methods,
provide the time-series measurement data stored in the storage device to an external processing apparatus in an external data description format,
cause the external processing apparatus, equipped with a machine learning computation device, to generate recommendation information including estimation of the state of a target organism and operational guidance based on past and current measurement data, analyze image information or audio information acquired from a user by an emotion estimation processing apparatus and optimize notification content based on the user's emotional state, and transmit the optimized notification content with the recommendation information to a terminal device, and
cause the terminal device to present the notification content.

### (Supplementary 2)

The system according to supplementary 1,
wherein the processor is configured to
process soil moisture information, temperature information, and light information included in the measurement data from the sensor unit.

### (Supplementary 3)

The system according to supplementary 1,
wherein the processor is configured to
generate recommendation information using a generative artificial intelligence model and receive a prompt sentence as an input.

### Application Example 2

### (Supplementary 1)

A system comprising a processor,
wherein the processor is configured to
acquire measurement information from an information acquisition device,
receive and store the measurement information in a storage device,
validate a format and reliability of the stored measurement information and supplement incomplete information,
analyze the stored measurement information to generate prediction information and advice information related to environment management,
adjust the prediction information and advice information based on an emotional state of a user using a notification adjustment unit, and
notify the user terminal device of the adjusted prediction information and advice information.

### (Supplementary 2)

The system according to supplementary 1,
wherein the processor is configured to
acquire measurement information from one or more sensing devices that detect a physical quantity.

### (Supplementary 3)

The system according to supplementary 1,
wherein the processor is configured to
analyze the stored measurement information using a machine learning technique or a generative information processing model.

What is claimed is:

1. A system comprising a processor;
wherein the processor is configured to:
receive measurement data from a sensor device configured to measure soil condition, water content, and light intensity;
store the measurement data in a database, analyze the stored data to generate predictions and advice related to crop management using an artificial intelligence module; and
notify a user terminal with the generated predictions and advice.

2. The system of claim 1, wherein the sensor device comprises a soil moisture sensor, a temperature sensor, and a light sensor.

3. The system of claim 1, wherein the processor is further configured to verify the format and consistency of the received data and to store the verified data in the database.
